(19) United States
(12) Patent Application Publication
(10) Pub. No.: US 2021/0020950 A1
(43) Pub. Date: Jan. 21, 2021

(54) NEGATIVE ELECTRODE FOR POWER GENERATION BATTERY, GASTRIC ACID BATTERY, METAL ION SECONDARY BATTERY, SYSTEM, AND METHOD FOR USING BATTERY

(71) Applicant: TOHOKU UNIVERSITY, Sendai-shi (JP)

(72) Inventor: Itaru HONMA, Sendai-shi (JP)

(73) Assignee: TOHOKU UNIVERSITY, Sendai-shi (JP)

(21) Appl. No.: 17/039,608

(22) Filed: Sep. 30, 2020

Related U.S. Application Data
(63) Continuation of application No. PCT/JP2019/022394, filed on Jun. 5, 2019.

(30) Foreign Application Priority Data
Jun. 20, 2018 (JP) 2018-117085
Feb. 27, 2019 (JP) 2019-034587

(51) Int. Cl. H01M 4/62; H01M 4/38; H01M 10/36; H01M 10/44; H01M 6/34; H01M 4/06; H02J 7/00

(52) U.S. Cl. CPC H01M 4/628; H01M 4/38; H01M 4/622; H01M 4/625; H01M 2004/027; H01M 10/44; H01M 6/34; H01M 4/06; H02J 7/0063; H01M 10/36

(57) ABSTRACT

A negative electrode for a power generation battery which is capable of suppressing hydrogen generation and a reduction in battery performance and performing stable power generation for a long time, a gastric acid battery, a metal ion secondary battery, a system, and a method for using a battery are provided. A mixture containing: a negative electrode powder made of a metal, an alloy, or a compound having a standard electrode potential lower than the standard hydrogen electrode potential; and a conductive polymer. The negative electrode powder consists of a zinc or magnesium powder, for example. The conductive polymer contains an acrylic resin or an epoxy resin and a conductive aid, for example. The mixture contains 60 to 90% by weight of the negative electrode powder, with the remainder being the conductive polymer and the conductive aid.

### FIELD OF THE INVENTION

The present invention relates to a negative electrode for a primary or secondary battery.

### DESCRIPTION OF RELATED ART

The present invention is a technique particularly suitable for a battery problematic in terms of hydrogen generation or a battery requiring long-term power generation. A battery to be used for a swallowable sensing device will be described as an example, as follows.

Conventionally, a swallowable sensing device required to have high biocompatibility and be miniaturized has used a button battery as a power source. In recent years, a gastric acid battery using gastric acid as an electrolytic solution has been developed as a new power source instead of a button battery. An example thereof is a gastric acid battery, in which a positive electrode desirably made of platinum and a cathode desirably made of zinc are installed on the inner surface of a cylindrical case into which gastric acid is introduced (for example, see Patent Document 1).

The gastric acid battery does not use any electrolytic solution harmful to the human body and uses a user's own body fluid, so as to pose no risk of damage to a gastrointestinal fluid due to leakage. Further, because of its relatively simple structure, the size can be reduced. Further, a metal having no adverse effect on the human body even when ingested is used as an electrode material, so that biocompatibility can be enhanced. Furthermore, materials having a low environmental load are used for electrodes and the structural body, and thus the battery can be directly flushed to the sewage after discharge without being collected.

### CITATION LIST

### Patent Literature 1: JP-A-2007-200739

### SUMMARY OF THE INVENTION

However, the conventional gastric acid electrode described in Patent Literature 1 is problematic in that gastric acid which is an acidic aqueous solution is electrolyzed to generate hydrogen upon power generation since the standard electrode potential of a negative electrode material such as zinc is lower than the standard hydrogen electrode potential. Such a gastric acid electrode is also problematic in that the thus generated hydrogen adsorbs onto the surface of the negative electrode to cover the surface as bubbles, reducing battery performance such as the power generation potential and the power generation capacity in a short time and making it impossible to perform stable power generation.

The present invention has been made in view of such a problem, and the purpose of the present invention is to provide a negative electrode for a power generation battery, and a power generation battery, which are capable of suppressing hydrogen generation and a reduction in battery performance, and generating power stably for a long time.

In order to achieve the above purpose, the present inventors have examined retroactively a mechanism of hydrogen generation upon power generation when the standard electrode potential of a negative electrode material is lower than the standard hydrogen electrode potential, such as when zinc is used as the negative electrode material. First, it is known that an electrode reaction at a negative electrode occurs with a local battery mechanism as in the case of corrosion, and the negative electrode material undergoes an electrochemical oxidation reaction due to the non-uniformity of the negative electrode material surface. For example, when a negative electrode material is zinc, zinc ions dissolve into an aqueous electrolyte according to formula (1).

Zn→Zn₂⁺+2e⁻ (1)

Next, electrons generated in formula (1) move to a place with a more positive potential, and react with protons in an electrolytic solution at the place to generate hydrogen molecules according to formula (2).

2H+2e⁻→H² (2)

The reactions of formulas (1) and (2) usually proceed at different places.

The present inventor has considered that hydrogen generation can be suppressed by suppressing the reaction of formula (2), and thus have completed the present invention. There has been no invention that has been completed by retroactively examining such a mechanism of hydrogen generation to suppress hydrogen generation.

Specifically, the negative electrode material according to the present invention is characterized by containing a negative electrode powder made of a metal, an alloy or a compound having a standard electrode potential lower than the standard hydrogen electrode potential, and a conductive polymer, wherein the conductive polymer is distributed in such a manner that the negative electrode powder is dispersed and fixed. The negative electrode material for an aqueous power generation battery according to the present invention preferably consists of a mixture containing the negative electrode powder and the conductive polymer.

Further, the problem of the present invention can be resolved by setting the proportion of the negative electrode powder constituting the negative electrode material to range from about 60% by weight to about 90% by weight, and using a conductive acrylic resin and a conductive aid as the remainder.

The negative electrode material according to the present invention can greatly suppress hydrogen generation.

The inventors believe that this is due to the following mechanism.

The negative electrode of the present invention comprises the conductive polymer existing around the negative electrode powder, and thus electrons generated by the reaction of zinc in the above formula (1) form hydrogen radicals in the process of reacting with protons. Since the hydrogen radicals have a property of being absorbed by the conductive polymer, the hydrogen radicals are absorbed by the conductive polymer after generation, but before being converted into hydrogen molecules. Therefore, the inventors believe that the reaction of the above formula (2) could be suppressed.

With the negative electrode material of the present invention, hydrogen generation can be suppressed, and a reduction in battery performance because of adsorption of hydrogen to the negative electrode and the surface covered by bubbles can also be suppressed.

Further, the negative electrode material of the present invention has a structure in which powders are close to each other or in contact with voids of the conductive polymer in the conductive polymer. Hence, percolation takes place such that an electrolytic solution gradually penetrates to the inside as it reacts with the powder. This can cause all of the negative electrode powder to function as an electrochemically activated negative electrode. Therefore, when used for a negative electrode, the negative electrode material for a power generation battery according to the present invention can efficiently generate power with respect to the weight ratio of the powder, and can perform stable power generation for a long time. Further, in the negative electrode material for a power generation battery according to the present invention, since the reaction of formula (2) is suppressed, electrons generated in formula (1) move from the negative electrode side to the positive electrode side via the conductive polymer. Accordingly, battery performance such as battery capacity and electromotive force can be improved.

In the negative electrode material for a power generation battery according to the present invention, the powder fixed to the conductive polymer flows out as metal ions into the electrolytic solution as it reacts with the electrolytic solution, and micro-voids are generated in the conductive polymer as the powder flows out. When charging is performed with an external charging power source in this state, and then metal ions are deposited in the micro-voids of the conductive polymer, the metal can be incorporated into the negative electrode again and the resultant can also be used as a negative electrode material for a secondary battery.

The negative electrode material for a power generation battery according to the present invention can be conveniently produced by compositing the negative electrode powder and the conductive polymer to form a composite material.

In the negative electrode material for a power generation battery according to the present invention, the negative electrode powder may be any metal, alloy, or compound as long as it has a standard electrode potential lower than the standard hydrogen electrode potential and high biocompatibility. The negative electrode powder is, for example, a powder of zinc (Zn), magnesium (Mg), calcium (Ca), iron (Fe), or triiron tetroxide (Fe₃O₄).

In the negative electrode material for a power generation battery according to the present invention, the conductive polymer preferably contains an acrylic resin or an epoxy resin and a conductive aid. In this case, hydrogen generation can be particularly effectively suppressed. The conductive aid is, for example, carbon black or acetylene black which is a kind thereof.

The negative electrode for a power generation battery according to the present invention can also be constituted by applying the negative electrode material for a power generation battery according to the present invention to the electrode surface. Further, the power generation battery according to the present invention consists of a positive electrode and the negative electrode according to the present invention.

The negative electrode for a power generation battery according to the present invention and the power generation battery according to the present invention can suppress hydrogen generation and a reduction in battery performance, and can perform stable power generation for a long time because of the negative electrode material for a power generation battery according to the present invention.

Further, the negative electrode material of the present invention can also be used as a negative electrode material for a secondary battery through the use of voids formed as the powder reacts and flows out.

According to the present invention, it is possible to provide a negative electrode for a power generation battery and a power generation battery, which are capable of suppressing hydrogen generation and a reduction in battery performance and capable of performing stable power generation for a long time.

### BRIEF DESCRIPTION OF THE DRAWINGS

FIG. 1 (a) shows a scanning electron microscope (SEM) photograph and FIG. 1 (b) a SEM photograph showing an enlarged view of a part of FIG. 1 (a) of the negative electrode material of an example prepared using a Zn powder and a conductive acrylic resin; that is, the negative electrode material for a power generation battery according to an embodiment of the present invention.

FIG. 2 (a) shows a SEM photograph of the surface of the negative electrode material of the example shown in FIG. 1 (a)-FIG. 1 (b) after immersion in a simulated gastric fluid for 72 hours, FIG. 2 (b) a SEM photograph showing an enlarged view of a part of FIG. 2 (a), and FIG. 2 (c) a SEM photograph of the surface of a Zn foil as a comparative sample after immersion in the simulated gastric fluid for 72 hours.

FIG. 3 (a)-FIG. 3 (b) shows the results of a constant-current discharge test using the negative electrode material of the example shown in FIG. 1 (a)-FIG. 1(b) as a negative electrode, specifically, FIG. 3 (a) a graph showing changes over time in voltage (Cell voltage) and FIG. 3 (b) a graph showing the relationship between electrical quantity (Capacity) and voltage (Cell voltage).

FIG. 4 (a) shows a SEM photograph of the cross section after the constant-current discharge test shown in FIG. 3 (a)-FIG. 3 (b) a SEM photograph showing an enlarged view of a part of FIG. 4 (a) of the negative electrode material of the example shown in FIG. 1 (a)-FIG. 1 (b).

FIG. 5 is a SEM photograph of the negative electrode material of an example produced using a Zn powder and a conductive polymethyl methacrylate (PMMA) resin; that is, the negative electrode material for a power generation battery according to an embodiment of the present invention.

FIG. 6 (a)-FIG. 6 (b) shows the results of a constant-current discharge test using the negative electrode material of the example shown in FIG. 5 as a negative electrode, specifically, FIG. 6 (a) a graph showing changes over time in voltage (Cell voltage) and FIG. 6 (b) a graph showing the relationship between electrical quantity (Capacity) and voltage (Cell voltage).

FIG. 7 (a)-FIG. 7 (b) shows the results of a constant-current discharge test using the negative electrode material of an example produced using a Zn powder and a conductive epoxy resin as a negative electrode; that is, the negative electrode material for a power generation battery according to an embodiment of the present invention, specifically, FIG. 7 (a) a graph showing changes over time in voltage (Cell voltage), and FIG. 7 (b) a graph showing the relationship between electrical quantity (Capacity) and voltage (Cell voltage).

### DETAILED DESCRIPTION OF THE INVENTION

Hereinafter, embodiments of the present invention will be described on the basis of the drawings and examples.

The negative electrode material for a power generation battery according to an embodiment of the present invention consists of a mixture containing a negative electrode powder made of a metal, an alloy or a compound having a standard electrode potential lower than the standard hydrogen electrode potential, and a conductive polymer. The negative electrode powder is, for example, a powder of zinc (Zn), magnesium (Mg), calcium (Ca), iron (Fe), or triiron tetroxide (Fe₃O₄). The conductive polymer contains, for example, an acrylic resin or an epoxy resin and a conductive aid.

The present inventor has studied the mixing proportion of a negative electrode powder of a negative electrode material as a negative electrode that solves the problem of the present application and the conductive polymer. The present inventor has confirmed that a negative electrode that solves the problem of the present application can be produced by setting the proportion of a negative electrode powder constituting the negative electrode material to range from about 60% by weight to about 90% by weight with the remainder being a conductive acrylic resin and a conductive aid.

Hereinafter, preferred embodiments for solving the problem of the present application will be specifically described.

### Example 1

A negative electrode for a power generation battery according to an embodiment of the present invention was prepared using a zinc (Zn) powder (Wako Pure Chemical Industries, Ltd.) as a negative electrode powder, and a conductive acrylic resin (room-temperature-curable acrylic resin for embedding SEM samples, KM-CO; PRESI) as a conductive polymer. First, after mixing and stirring the Zn powder and the conductive acrylic resin, the mixture was applied to the tip of a gold wire and then dried under ordinary temperature and pressure for about 24 hours. After drying, the resultant was cut into a desired shape and size, and washed with ethanol to remove unbound Zn and resin, thereby obtaining a negative electrode material. The mixing proportions of the Zn powder and the conductive acrylic resin are about 75% by weight and about 25% by weight, respectively. Further, the conductive acrylic resin contains carbon as a conductive aid.

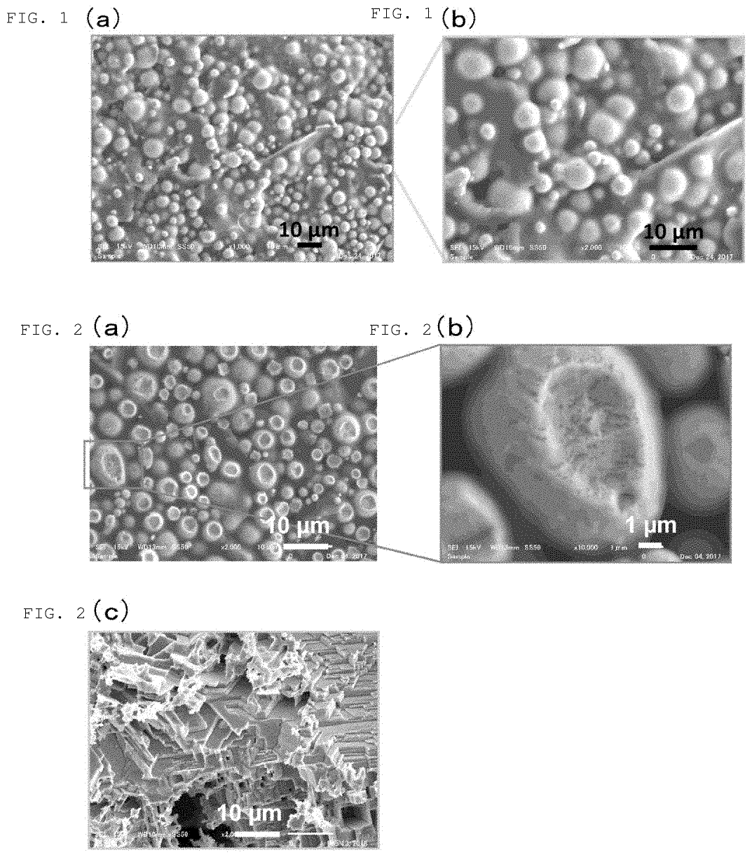

FIG. 1 shows a scanning electron microscope (SEM) photograph of the prepared negative electrode material (hereinafter, referred to as “negative electrode material of Example 1”). As shown in FIG. 1, it was confirmed that the conductive acrylic resin was distributed so as to disperse and fix Zn particles having a diameter of about 1 to 5 μm, and the conductive acrylic resin bound Zn powders together to form a composite.

### [Immersion Test in Simulated Gastric Fluid]

Using the negative electrode material of Example 1, an immersion test in a simulated gastric fluid (First fluid for the disintegration test, pH 1.2; Kanto Chemical Co., Inc.) was conducted. In this test, the negative electrode material of Example 1 was formed into a plate having a size of 5 mm×5 mm×0.5 mm, and was placed in a 4-ml screw tube together with the simulated gastric fluid while avoiding the intrusion of air. After immersion in the simulated gastric fluid for 72 hours, the volume of generated bubbles (hydrogen) was measured. For comparison, a 5 mm×5 mm×0.1 mm Zn foil (Nilaco Corporation) was immersed in the simulated gastric fluid in the same manner as the negative electrode material of Example 1, and the volume of hydrogen bubbles after 72 hours of immersion was measured.

The measured amount of hydrogen generated was 2.0 ml for the Zn foil as a comparative sample and 0.21 ml for the negative electrode material of Example 1. Thus, the amount of hydrogen generated in the case of the negative electrode material of Example 1 was about 1/10 of that of the Zn foil, suggesting that hydrogen generation was successfully suppressed. As described above, it is considered that unlike the Zn foil, the negative electrode material of Example 1 has a conductive polymer existing around the material, preventing protons from becoming hydrogen molecules and decreasing the amount of hydrogen generated.

FIGS. 2(a) and (b) show SEM photographs of the surface of the negative electrode material of Example 1 after immersion in the simulated gastric fluid for 72 hours. FIG. 2(c) shows a SEM photograph of the surface of the Zn foil as a comparative sample after immersion in the simulated gastric fluid for 72 hours. As shown in FIG. 2(c), it was confirmed that corrosion progressed over the entire surface of the Zn foil. On the other hand, as shown in FIGS. 2(a) and (b), it was confirmed in the negative electrode material of Example 1 that corrosion progressed from sites where Zn was exposed on the surface of the conductive polymer.

### [Constant-Current Discharge Test]

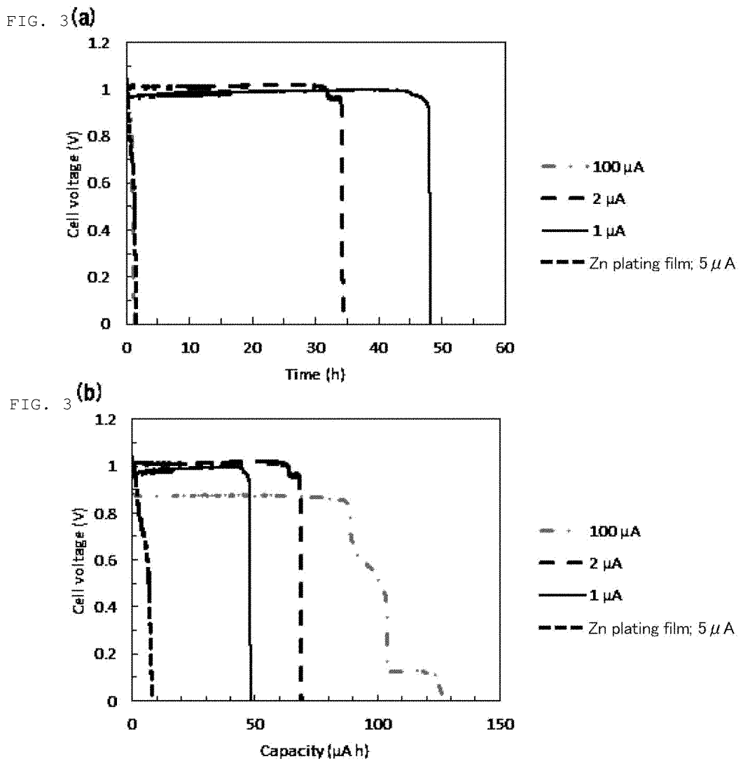

A constant-current discharge test was conducted using the negative electrode material of Example 1. In this test, in order to simulate a gastric acid battery in a swallowable sensing device, which is a preferred application example described above, a laminate cell was prepared using an Ag/AgCl electrode as a positive electrode, the negative electrode material of Example 1 as a negative electrode, an Ag/AgCl electrode as a reference electrode, and simulated gastric acid (First fluid for the disintegration test, pH 1.2; Kanto Chemical Co., Inc.) as an electrolytic solution. The negative electrode material of Example 1 had a size of about 2 mm×2 mm×1 mm. This cell was connected to a potentiostat (VMP3 Bio-Logic; TOYO Corporation), and then a discharge test was conducted at discharge current values of 1 μA, 2 μA, and 100 μA. For comparison, a similar test was conducted on materials prepared using a Zn plating film (about 2 mm×2 mm×1 mm) and a Zn foil as negative electrodes. The Zn plating film is a film formed by electroplating Zn on the surface of a Si substrate on which Ti and Au have been formed. Changes over time in voltage (Cell voltage) and the relationship between electrical quantity (Capacity) and voltage (Cell voltage) are shown in FIGS. 3 (a) and (b), respectively, in the discharge test.

As shown in FIG. 3(a), the Zn plating film of the comparative example (current value: 5 μA) exhibited a voltage drop within several hours, as indicated with a dark dashed line. This is because, with hydrogen generation, the electrode surface was covered by bubbles to increase the internal resistance. When the discharge current value was 100 μA, as indicated with a thin alternate long and short dashed line, the negative electrode material of Example 1 was confirmed to exhibit a voltage drop due to an increase in internal resistance as in the case of the Zn plating film and Zn foil (not shown) of the comparative examples. On the other hand, when the discharge current value was 1 μA as indicated with a solid line and the discharge current value was 2 μA as indicated with a thin dashed line, the voltage of about 1 V was confirmed to be maintained for 30 hours or more. From this result, when the discharge current value was 100 μA, the reaction of the negative electrode material of Example 1 proceeded too fast at the electrode, so that the speed of absorption of the generated hydrogen radicals by the conductive polymer cannot keep up therewith. Hence, hydrogen generation occurred, and the bubbles covered the electrode surface to increase the internal resistance. However, when the discharge current value was 1 μA or 2 μA, the reaction at the electrode proceeded gently, and the generated hydrogen radicals were absorbed by the conductive polymer without any problem, so as to be able to prevent hydrogen generation.

FIG. 3(b) shows the relationship between electrical quantity and voltage in the discharge test shown in FIG. 3(a). As shown with a thin alternate long and short dashed line, when the discharge current value was 100 μA, the voltage dropped to about 0.9 V at 10 μAh, but after that, 0.9 V could be maintained, and the electrical quantity until the voltage sharply dropped again was about 86 μAh. When the discharge current value was 1 μA as indicated with a solid line and the same was 2 μA as indicated with a thin dashed line, 1 V was continuously maintained, and the electrical quantities at which the voltage sharply dropped were about 48 μAh and about 70 μAh, respectively. On the other hand, regarding the Zn foil (not shown) and the Zn plating film (when the discharge current value was 5 μA) indicated with a dark dashed line, the Zn plating film exhibited a sharp voltage drop at an electrical quantity of 10 μAh or less. Also, when comparing the amount of power generation, it was confirmed that when the negative electrode material of Example 1 was used, a large amount of power generation was obtained as compared with the Zn foil or the Zn plating film. In the case of using the negative electrode material of Example 1, the amount of power generation equivalent to the theoretical capacity found from the AgCl amount of the positive electrode was obtained. Specifically, at least the amount of power generation sufficient for the completion of the reaction of the positive electrode was obtained. Thus, a higher amount of power generation can be expected depending on the selection of a positive electrode.

The reason why a high amount of power generation is obtained with the negative electrode material of Example 1 is that when the discharge current value is 1 μA or 2 μA, as described above, the amount of hydrogen generated is suppressed as compared with the Zn plating film and the Zn foil, and the electrolytic solution gradually penetrates to cause efficient reaction with the Zn powder. Further, a voltage drop by 0.9 V at 10 μAh when the discharge current value of the negative electrode material of Example 1 was 100 μA took place because the speed of absorption of hydrogen radicals by the conductive polymer could not keep up with the speed of reaction, so as to result in hydrogen generation, and thus the voltage could not be maintained at 1 V. However, even in this case, although hydrogen generation occurred, the amount of power generation was overwhelmingly higher than that of the Zn plating film and the Zn foil. This may be because the electrolytic solution penetrated continuously to the inside even though a violent reaction occurred on the negative electrode surface, and thus the Zn powder reacted efficiently.

FIG. 4 shows a SEM photograph of the cross section of a negative electrode controlled with a discharge current value of 2 μA among negative electrodes consisting of the negative electrode material of Example 1 after the constant-current discharge test.

As shown in FIG. 4(b), many spherical voids were observed near the surface of the negative electrode in contact with the simulated gastric fluid (right side in the figure). Since these voids are of almost the same size as the Zn particles, it is considered that the voids are traces of the dissolution of the Zn particles by the simulated gastric fluid. On the other hand, it was confirmed that spherical Zn particles were present inside the negative electrode. In the vicinity of the boundary between a region where the spherical Zn particles existed and a region in the vicinity of the surface where spherical voids were observed, non-spherical and distorted Zn particles were observed. Since voids were recognized between the distorted Zn particles and the surrounding conductive acrylic resin, they were considered to be Zn in the process of being dissolved by the simulated gastric fluid. From this result, it is considered that in the simulated gastric fluid, the reaction with the simulated gastric fluid gradually proceeded from the surface of the negative electrode, and specifically, the simulated gastric fluid gradually penetrates into the negative electrode while forming voids through dissolution of Zn particles.

As described above, the negative electrode material of Example 1 of the present invention suppresses hydrogen generation and can maintain stable voltage for a long time, as well as an electrolytic solution penetrates into the inside while reacting with Zn particles. Hence, it can be understood that since the Zn powder reacts efficiently, the negative electrode material has a feature of obtaining a high amount of power generation.

### Example 2

The negative electrode material for a power generation battery of an embodiment of the present invention was prepared using a zinc (Zn) powder (Wako Pure Chemical Industries, Ltd.) as a negative electrode powder and a conductive polymethyl methacrylate resin as a conductive polymer. First, the Zn powder, a raw material of the polymethyl methacrylate (PMMA) resin, and acetylene black (Denka Company Limited; formerly Denki Kagaku Kogyo Kabushiki Kaisha.) as a conductive aid were mixed and stirred. The mixture was applied to the tip of a gold wire, and then dried at normal temperature for 24 hours and further dried for 24 hours at 40° C. After drying, it was cut into a desired shape and size, and then washed with ethanol to remove unbound Zn and resin, thereby obtaining a negative electrode material. Note that the mixing proportions of PMMA, the Zn powder, and the conductive aid are about 24.8% by weight, about 74.5% by weight, and about 0.7% by weight, respectively.

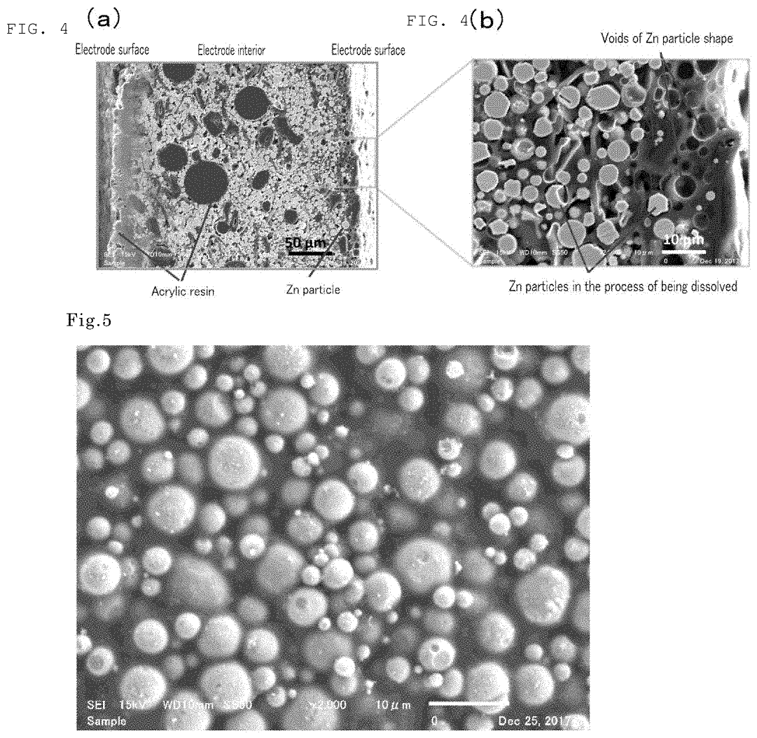

FIG. 5 shows a scanning electron microscope (SEM) photograph of the prepared negative electrode material (hereinafter, referred to as “the negative electrode material of Example 2”). As shown in FIG. 5, it was confirmed that the conductive PMMA resin is distributed so as to cover the Zn particles having a diameter of about 1 to 5 μm, and the conductive PMMA resin binds Zn powders together to form a composite.

### [Immersion Test in Simulated Gastric Fluid]

Using the negative electrode material of Example 2, an immersion test in a simulated gastric fluid (First fluid for the disintegration test, pH 1.2; Kanto Chemical Co., Inc) was conducted. In this test, the negative electrode material of Example 2 was formed into a plate having a size of 5 mm×5 mm×0.5 mm, and was placed in a 4-ml screw tube together with the simulated gastric fluid while avoiding the intrusion of air. After 48 hours of immersion in the simulated gastric fluid, the volume of generated bubbles (hydrogen) was measured. For comparison, a 5 mm×5 mm×0.5 mm Zn foil was similarly immersed in the simulated gastric fluid, and the volume of hydrogen bubbles after 48 hours of immersion was measured.

The measured amount of hydrogen generated was 2.7 ml for the Zn foil as the comparative sample and 0.3 ml for the negative electrode material of Example 2. Thus, the amount of hydrogen generated in the case of the negative electrode material of Example 2 was about 1/10 of that of the Zn foil. It was thus considered that hydrogen generation could be suppressed as in Example 1.

### [Constant-Current Discharge Test]

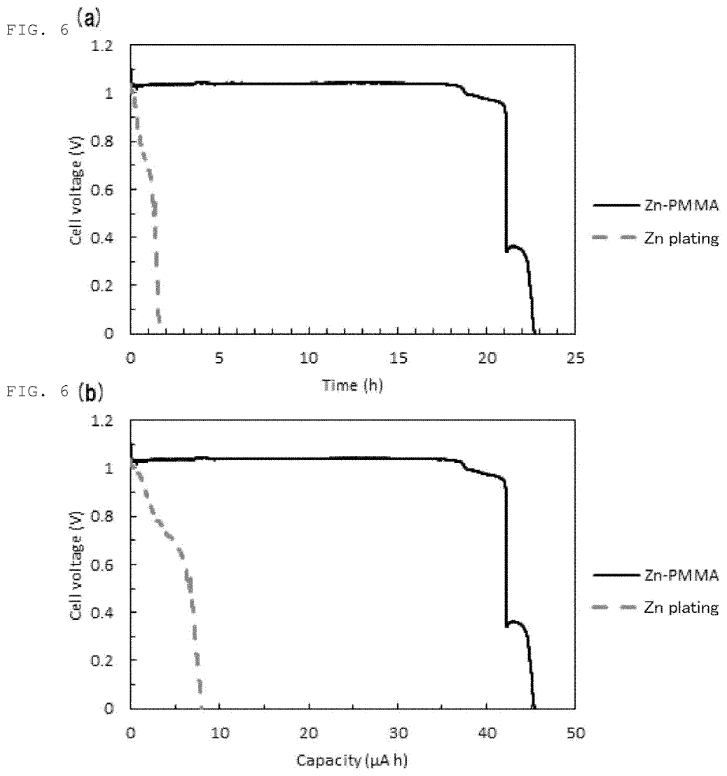

A constant-current discharge test was conducted using the negative electrode material of Example 2. The test was the same as Example 1 except that the negative electrode used was the negative electrode material of Example 2 (about 2 mm×2 mm×1 mm) and the discharge current value was 5 μA. For comparison, a similar test was conducted on a negative electrode prepared using a Zn plating film (the preparation method and size were the same as those in Example 1). FIGS. 6(a) and (b) show changes over time in the voltage (Cell voltage) and the relationship between electrical quantity (Capacity) and voltage (Cell voltage), respectively, in the discharge test.

As shown in FIG. 6(a), the Zn plating film was confirmed to exhibit a voltage drop, as indicated with a thin dashed line, due to an increase in the internal resistance associated with hydrogen generation when the discharge current value was 5 μA. In contrast, the negative electrode material of Example 2 was confirmed to maintain a voltage of about 1 V, as indicated with a solid line, when the discharge current value was 5 μA. FIG. 6(b) further shows the relationship between the amount of power generation and voltage in the discharge test of FIG. 6(a). As shown with the solid line, the negative electrode material of Example 2 exhibited a sharp voltage drop when the electrical quantity was about 40 μAh while maintaining 1 V at 5 μA. On the other hand, the Zn plating film of the comparative example was unable to maintain voltage and exhibited a sharp voltage drop when the electrical quantity was 10 μAh or less. As described above, even when the conductive polymer is changed as in Example 2, the amount of hydrogen generated is suppressed, the stable voltage is maintained for a long time, and a high amount of power generation can be obtained in the case of the negative electrode material of the present invention as compared with the Zn plating film.

### Example 3

A negative electrode material for an in vivo swallowable power generation battery of an embodiment of the present invention was prepared using a zinc (Zn) power (Wako Pure Chemical Industries, Ltd.) as a negative electrode powder and a conductive epoxy resin as a conductive polymer. First, the Zn powder, a raw material of an epoxy resin (Nissin EM Co., Ltd.) and acetylene black (Denka Company Limited; formerly Denki Kagaku Kogyo Kabushiki Kaisha) as a conductive aid were mixed and stirred, and then the mixture was applied to the tip of a gold wire, followed by vacuum drying at 70° C. for 18 hours. After drying, it was cut into a desired shape and size, and washed with ethanol to remove unbound Zn and resin, thereby obtaining a negative electrode material. Note that the mixing proportions of the epoxy resin, the Zn powder, and the conductive aid are about 36.9% by weight, about 60.9% by weight, and about 2.2% by weight, respectively.

### [Constant-Current Discharge Test]

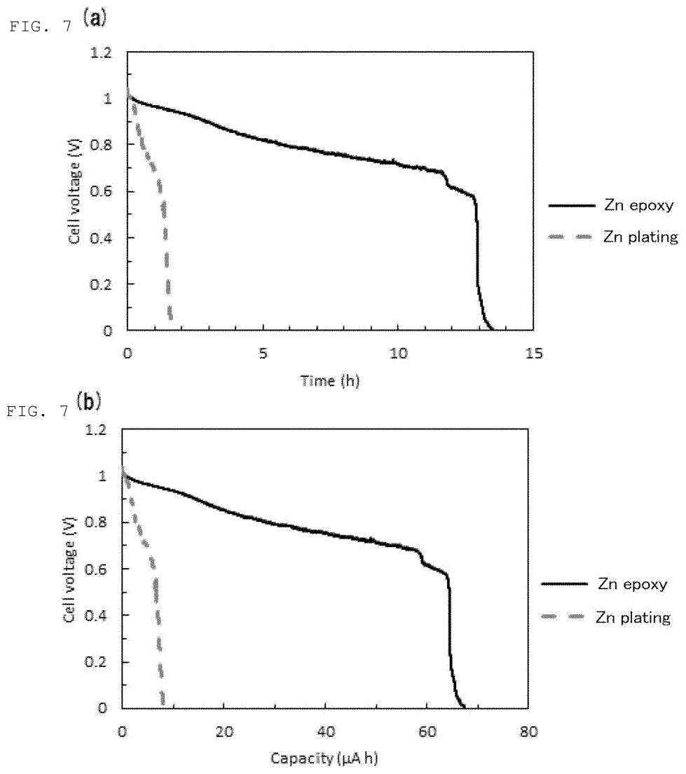

Using the prepared negative electrode material (hereinafter, referred to as “the negative electrode material of Example 3”), a constant-current discharge test was conducted. This test was conducted in the same manner as in Example 1 except that the negative electrode material of Example 3 (about 2 mm×2 mm×1 mm) was used as the negative electrode, and the discharge current value was 5 μA. For comparison, a similar test was conducted on a negative electrode prepared using a Zn plating film (the preparation method and size were the same as those in Example 1). FIGS. 7(a) and (b) show changes over time in voltage (Cell voltage) and the relationship between electrical quantity (Capacity) and voltage (Cell voltage) in the discharge test, respectively.

As shown in FIG. 7 (a), it was confirmed that whereas the Zn plating film exhibited, as indicated with a thin dashed line, a voltage drop due to an increase in internal resistance associated with hydrogen generation when a discharge current value was 5 μA, the negative electrode material of Example 3 exhibited, as indicated with a solid line, a gradual voltage drop over time when a discharge current value was 5 μA, but the voltage of about 1 to 0.6 V was obtained. FIG. 7(b) shows the relationship between electrical quantity and voltage in the discharge test of FIG. 7(a). As indicated with the solid line, the negative electrode material of Example 3 exhibited a gradual voltage drop at 5 μA, while exhibiting a sharp decrease in electrical quantity by about 60 μAh. On the other hand, the electrical quantity of such a sharp decrease in the case of the Zn plating film of the comparative example was 10 μAh or less. As described above, even the negative electrode material of Example 3 can achieve the suppressed amount of hydrogen generated and the obtainment of a high amount of power generation as compared with the Zn plating film.

### [Use of Negative Electrode for Secondary Battery]

An electrode manufactured using the negative electrode material of the present invention has a structure where an electrolytic solution in which particles are close to each other undergoes percolation. The electrolytic solution gradually penetrates to the inside as it reacts with the powder, and, portions of the particles remain as microvoids, as the particles dissolve.

On the other hand, when charging is performed with an external charging power source in this state, metal ions are deposited in the micro-voids of the conductive polymer, making it possible again to incorporate metals in the negative electrode, and to use it as an electrode for a metal ion secondary battery.

According to the present invention, as described above, a negative electrode of a metal ion secondary battery can be produced simply by compositing particles with a conductive polymer, and an inexpensive and highly functional negative electrode of a secondary battery can be provided.

As shown in the results of each of the above tests, the negative electrode material for a power generation battery of the embodiment(s) of the present invention contains not only a negative electrode powder but also a conductive polymer, so that the process in which protons are converted to hydrogen molecules is inhibited. Therefore, hydrogen generation can be suppressed. For this reason, it is possible to prevent the electrode surface of the negative electrode from being covered by bubbles or to suppress a reduction in battery performance due to the adsorption of hydrogen. Further, because of the percolation structure, in which an electrolytic solution gradually penetrates to the inside as it reacts with the powder, the powder reacts efficiently. Hence, the negative electrode material is capable of generating a high amount of power, and can perform stable power generation for a long time.

Further, metal ions are deposited again at places where the metal powder is dissolved, it can be used as a metal ion secondary battery.

The negative electrode material for a power generation battery according to the embodiment(s) of the present invention can suppress hydrogen generation by being used for the negative electrode of the battery. Hence, when the negative electrode material is used in an in vivo swallowable battery, the battery can be used as a battery capable of performing stable power generation. It can also be used as an inexpensive negative electrode for a high-capacity secondary battery having a high energy density, such as a zinc-air battery or a zinc secondary battery. Further, since such a secondary battery can be used as a power source for various moving objects such as electric vehicles, hybrid vehicles, robots, and drones, it can also be used as a substitute for a lithium ion battery.

1. A negative electrode for an aqueous power generation battery, which contains
a negative electrode material consisting of: a negative electrode powder made of a metal, an alloy or a compound having a standard electrode potential lower than the standard hydrogen electrode potential; and a conductive polymer, wherein
the conductive polymer is distributed in such a manner that the negative electrode powder is dispersed and fixed, and
the negative electrode material contains the negative electrode powder in a proportion of 60% by weight or more and 90% by weight or less.

2. The negative electrode for an aqueous power generation battery according to claim 1, wherein an electrolytic solution penetrates into the negative electrode as the electrolytic solution reacts with the negative electrode powder.

3. The negative electrode for an aqueous power generation battery according to claim 1, wherein the conductive polymer contains carbon as a conductive aid.

4. The negative electrode for an aqueous power generation battery according to claim 1, wherein the negative electrode powder is made of a zinc or magnesium powder.

5. The negative electrode for an aqueous power generation battery according to claim 1, wherein the conductive polymer contains an acrylic resin or an epoxy resin, and a conductive aid.

6. A gastric acid battery, comprising a positive electrode and the negative electrode according to claim 1,

7. A metal ion secondary battery, comprising a positive electrode and the negative electrode according to claim 1.

8. A system, comprising: a power generation battery comprising a positive electrode and the negative electrode according to claim 1;
and a control device for controlling a discharge current of the negative electrode, wherein
the control device controls with the discharge current that suppresses hydrogen generation from the negative electrode.

9. A method for using a battery, comprising:
a step of preparing a battery containing a positive electrode and a negative electrode that consists of a negative electrode powder made of a metal, an alloy or a compound having a standard electrode potential lower than the standard hydrogen electrode potential, and a conductive polymer, wherein the conductive polymer is distributed in such a manner that the negative electrode powder is dispersed and fixed;
a step of immersing the positive electrode and the negative electrode of the battery in an acidic electrolytic solution; and
a step of generating power while suppressing hydrogen generation through the reaction on the negative electrode.
